(19) United States
(12) Patent Application Publication
(10) Pub. No.: US 2022/0339314 A1
(43) Pub. Date: Oct. 27, 2022

(54) DISINFECTING AIR FILTRATIONS SYSTEM CONFIGURATION

(71) Applicant: Healthway Home Products Company Inc., Pulaski, NY (US)

(72) Inventors: Vincent G. Lobdell, Pulaski, NY (US); Mitchell Raymond Lobdell, Constantia, NY (US); Xuchen Wang, Pulaski, NY (US); Kevin GUERNSEY, Constantia, NY (US); Alexander Thomas Frechette, Mexico, NY (US); Timothy George Cusack, Central Square, NY (US); Brooke Alan Rowe, Vernon, NY (US)

(21) Appl. No.: 17/729,726

(22) Filed: Apr. 26, 2022

Related U.S. Application Data
(60) Provisional application No. 63/180,471, filed on Apr. 27, 2021.

(51) Int. Cl. A61L 9/22; B03C 3/011; B03C 3/155; B03C 3/36; B03C 3/68

(52) U.S. Cl. CPC A61L 9/22; B03C 3/011; B03C 3/155; B03C 3/36; B03C 3/68; A61L 2209/111; A61L 2209/14; B03C 2201/24

(57) ABSTRACT

Methods and apparatus of the present disclosure monitor and change operation of an air filtering system or apparatus dynamically over time. Changes to the air filtering apparatus may be associated with a type of facility and air purity requirements associated with the type of facility. Examples of different types of facilities include an office building, a clean room, and a hospital. Apparatus of the present disclosure may include conventional air filters and may include disinfecting air filter sub-assemblies that use a high voltage to charge particles in the air such that those particles may conglomerate and be captured more easily in an air. Methods consistent with the present disclosure may change an air flow rate or may change the voltage used to charge the air particles as conditions associated with the air of a facility change over time.

### CROSS-REFERENCE TO RELATED APPLICATIONS

The present application claims the priority benefit of U.S. provisional application No. 63/180,471 filed Apr. 27, 2021, the disclosure of which is incorporated herein by reference.

### BACKGROUND

### Field of the Disclosure

The present disclosure is generally related to filter assemblies for air purification systems. More specifically, the present disclosure is directed to controlling operation of an air filtration apparatus.

### Description of the Related Art

Various types of air filters have been made for many years. Conventional air filters commonly rely on a flow of air that passes through a filter, where the filter traps particles that are larger than a hole size associated with the filter. As the hole size of a filter decreases, an amount of resistance to the airflow increases. This means that blowers that circulate air through an air filtration apparatus must be more powerful to maintain a given airflow rate when denser filters are used. This increases an amount of air pressure that a blower must provide to maintain that airflow. This means that increasing an amount of filtering capability using smaller hole sizes will result in increased operating and/or manufacturing costs of an air filtering apparatus. This is because a blower may have to be provided with a larger amount of electrical power to maintain an air flow rate, this may force a manufacturer to replace a less powerful blower with a more powerful blower.

Another technique that has been used to filter air, is to charge particles in an air flow using a high electric voltage and then capture the charged particles on a surface that has a different or opposite charge. Such air filters are commonly referred to as ionizing or ionizer air purifiers. Ionizing air purifiers, however, generate ozone that is emitted into environments where people live and work. People that breathe in ozone commonly suffer from health effects that include chest pain, coughing, throat irritation, and congestion. Breathing ozone is also associated with various illnesses and increased rates of bronchitis, emphysema, and asthma.

With the emergence of new infections diseases caused by various pathogens (such as coronavirus COVID-19, antibiotic resistant bacteria, and antifungal resistant fungi), the need to filter very small particles out of the air has increased dramatically. The size of viruses range from 20 nanometers (nm) to about 5000 nm, where COVID-19 has a diameter of about 100 nm.

Air filtering apparatus are also used in different types of operating environments that include yet are not limited to buildings, hospitals, and clean rooms. Each of these different operating environments may each have a different set of air filtering requirements. For example, a clean room may require filtering all air in the clean room faster than air filtering requirements of a building. Current air filtering apparatus, however, do not adjust their operation as conditions associated with the air change. For example, in an instance when food is burned in a kitchen of a building, a conventional air filtering apparatus would not change its operation based on the presence of smoke in the air. What are needed a new methods and apparatus that allow or that force changes to operating conditions of an air filtering apparatus as conditions of air filtered by the air filtering apparatus changes.

### SUMMARY OF THE PRESENTLY CLAIMED INVENTION

The presently claimed invention is directed to an apparatus for filtering air and is directed to a method for making such an air filtering apparatus. In a first embodiment, a presently claimed method includes receiving sensor data, evaluating the received sensor data to identify a first set of operating conditions associated with the air filtration apparatus, accessing data that identifies operational requirements of an environment associated with the air filtration apparatus, and identifying that an operational setting of the air filter apparatus should be changed based on identifying that the first set of conditions do not correspond to the operational requirements of the environment. This method may also include the step of initiating a change to the operational setting of the air filtration apparatus. This change to the the environment may result in changing the first set of operating conditions associated with the air filtration apparatus.

In a second embodiment, the presently claimed method may be implemented as a non-transitory computer-readable storage medium where a processor executes instructions out of the memory. Here the method may also include receiving sensor data, evaluating the received sensor data to identify a first set of operating conditions associated with the air filtration apparatus, accessing data that identifies operational requirements of an environment associated with the air filtration apparatus, and identifying that an operational setting of the air filter apparatus should be changed based on identifying that the first set of conditions do not correspond to the requirements in the environment. This method may also include the step of initiating a change to the operational setting of the air filtration apparatus. This change to the operational setting may result in changing the first set of operating conditions associated with the air filtration apparatus.

In a third embodiment an apparatus may include a plurality of sensors that provide sensor data to a processor that executes instructions out of a memory. Here the processor may execute instructions out of the memory to receive sensor data, evaluate the received sensor data to identify a first set of operating conditions associated with the air filtration apparatus, access data that identifies operational requirements of an environment associated with the air filtration apparatus, and identify that an operational setting of the air filter apparatus should be changed based on identifying that the first set of conditions do not correspond to the operational requirements of the environment. The processor may also execute instructions out of the memory to initiate a change to the operational setting of the air filtration apparatus. This change to the operational setting may result in changing the operating conditions associated with the air filtration apparatus.

### DESCRIPTIONS OF THE DRAWINGS

FIG. 1 illustrates parts that may be included in a disinfecting air filtration apparatus.

FIG. 2 includes a controller that may be used to monitor air quality, contaminates in the air (e.g. smoke, particulate matter, bacteria, fungi, or viruses), levels of volatile organic compounds (VOCS), air particle counts, air flow rates, ozone levels, and/or pressure drops when voltages applied to disinfecting filters are controlled or adjusted.

FIG. 3 illustrates a series of steps that may be performed by a processor of an air filtration apparatus when operational characteristics of the air filtration apparatus are controlled over time.

FIG. 4 illustrates a series of steps that may be performed when operation of a disinfection filtration system (DFS) or filter apparatus is controlled.

FIG. 5 illustrates a computing system that may be used to implement an embodiment of the present invention.

### DETAILED DESCRIPTION

Methods and apparatus of the present disclosure monitor and change operation of an air filtering system or apparatus dynamically over time. Changes to the air filtering apparatus may be associated with a type of facility and air purity requirements associated with the type of facility. Examples of different types of facilities include an office building, a clean room, and a hospital. Apparatus of the present disclosure may include conventional air filters and may include disinfecting air filter sub-assemblies that use a high voltage to charge particles in the air such that those particles may conglomerate and be captured more easily in an air purification system. Methods consistent with the present disclosure may change an air flow rate or may change the voltage used to charge the air particles as conditions associated with the air of a facility change over time.

A disinfecting filtration system (DFS), also referred to as electrically enhanced filtration (EEF) is an air purification system that uses two mechanisms to maintain high air cleaning performance. An EEF air purification system may use high energy fields to facilitate the aggregation and capture of ultrafine particles. Such a system may effectively increase particle size by forming clusters ultrafine particles. Such a high energy field may be controlled in a manner that contains and captures charged particles without emitting charged particles from the filter system. Such a filtering process may be based on an “entry control grid” that is located before a front part of a main filter and a “rear control grid” (or “exhaust control grid”) that may be affixed to a rear part of the main filter. The entry ground control grid and the rear/exhaust control grid may be tied to an Earth ground connection that prevents these grids from be energized by the high energy field. Each of the entry control grid and rear/exhaust control grid may be a screen include holes that do not allow service personnel to reach into an energized portion of a disinfecting filtration system.

Even in instances where ions generated by the high energy field, such charged particles may be isolated in the main filter between the entry control grid and the rear/exhaust control grid on a rear side of the filter. The controlled, isolated high energy field generated by the EEF continually creates high energy exposure through pleats and fibers of a main filter creating microbiostasis (“prevention of organism growth”) in the main filter. This may prevent live organisms from escaping back into the air. These two mechanisms work together to provide the ultraclean filtration of particles as well as continual prevention of organism growth in the EEF filter.

A filtering apparatus may include pre-filters to remove larger particles. These pre-filters may increase the effective lifespan of electrically enhanced filters and reduce the load placed on a high voltage alternating current air conditioning (HVAC) system caused by pressure drops. Pre-filters should be replaced more frequently than the electrically enhanced filters, and failure to do so may limit the effectiveness of the air filtration system and increase the pressure drop load placed on the HVAC system. The replacement of pre-filters should be as simple a process as possible, and ideally require little to no expertise to do so. The ease of maintenance allows for timely replacement without requiring the expense and delay of service calls. Further, the replacement of pre-filters should not require a complete shutdown of the HVAC system in order to allow continuous filtration of the air being treated.

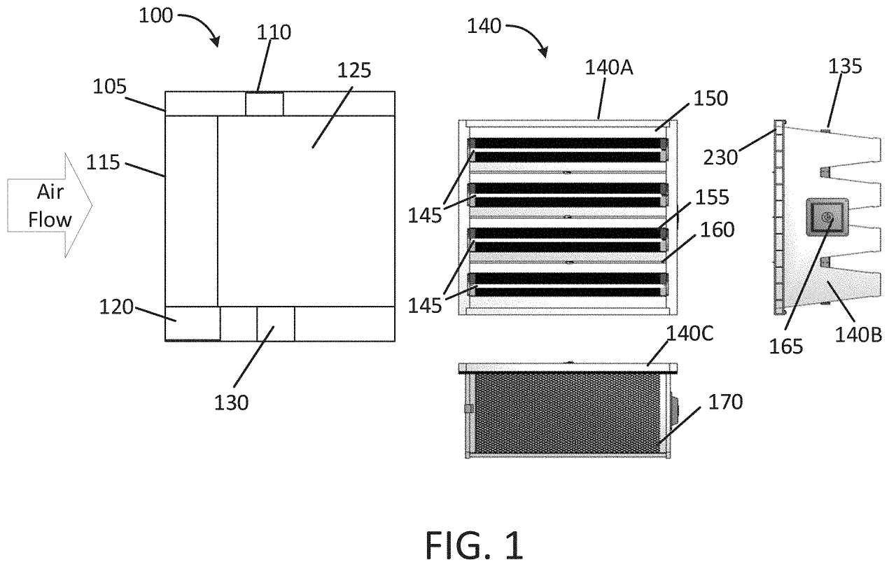

FIG. 1 illustrates parts that may be included in a disinfecting air filtration apparatus. The apparatus of FIG. 1 may include electronic circuits (i.e. a controller) that changes voltages provided to parts of the filtration apparatus 100 when air is filtered. The system 100 may include disinfecting filter assembly 105, power control unit 110, pre-filter 115, controller 120, disinfecting filter 125, and a ground contact 130. Disinfecting filter 125 may be type of filter referred to as a V-Bank filter 140. FIG. 1 includes several different views 140A, 140B, and 140C of V-Bank filter 140.

Filtration systems installed in a building may include several different filter assemblies 105 that are built as part of the building's high voltage alternating current (HVAC) air circulation system. A number of filter assemblies 105 included in such an array may only be limited by a configuration of an HVAC system into which disinfecting filter 125 assemblies is being incorporated into. A power supply may provide electrical power to the power control unit 110 in filter assembly 105 and power control unit 110 may provide power to a respective V-Bank filter 140 when a high-energy field is generated inside a disinfecting filter 125 such as V-Bank filter 140.

Pre-filter 115 may be a filter that captures large particles before they may enter the V-Bank filter 140. In certain instances, pre-filter 115 may be selected to capture particles larger than a particular size, for example, pre-filter 115 may be selected to provide a minimum efficiency reporting filtration value (MERV) rating of at least MERV 8. The minimum size of particles captured by the prefiltration process can vary depending upon a given application, a desired air flow, and/or a resistance to the air flow capacity of a particular high voltage air conditioning (HVAC) system. FIG. 1 includes a large arrow labeled “Air Flow,” this arrow illustrates air flowing into filter assembly 105 through pre-filter 115.

As mentioned above, FIG. 1 also includes three different views of V-Bank filter 140 included in filter assembly 100. These three different views include V-Bank filter front view 140A, a second V-Bank filter view 140B, and a third view of the V-Bank filter 140C. Parts that may be included within a V-Bank filter include high voltage wires 145, an entry control grid, a high energy transfer grid, ground bar 160, power contact pad 165, and a rear control grid 170. Items 150 and 155 identify locations respectively where an entry control grid and a high energy transfer grid may be located. The entry control grid may have a surface that is like a screen, chicken wire, or a plate perforated with holes that prevents a person from touching high energy wires 145 or other energized components (e.g. the high energy transfer grid) within the V-Bank filter. Rear control grid 170 may also have a surface like a screen, chicken wire, or perforated plate and as mentioned above the entry control grid and rear control grid may be grounded. The high energy transfer grid may be charged to a high voltage and this grid may be in close proximity to filter elements included in a V-Bank filter assembly.

Power may be routed to high energy wires 145 of a respective V-Bank filters from a respective power control unit 110 via power contact 165 and connecting wires or high energy transfer grids. Ground contact 130 may be used to provide an Earth ground connection 135 to a frame or electrical connector of the V-Bank filter 140.

Power control unit 110 may activate a high energy field by delivering a voltage to a to a high-voltage contact or wires connected to high energy wires 145. Voltages provided to the high energy wires 145 may be high enough to generate a high energy field within V-Bank filter 140. This high energy filed may be provided to filter media inside of V-Bank filter 140 such that an electric field gradient is generated between a high energy control grid and rear control grid 170. This field gradient may be generated based on charged particles transferring charge to the high energy control grid and may be based on the rear control grid 170 being grounded.

In certain instances, a V-Bank filter may user filter media that is a lesser dense media (for example 95 DOP) as compared to a standard HEPA filter (99.97 DOP). This may allow the filter media to have a higher gram holding weight and thus allow the filter media to hold more dust as compared to a standard HEPA filter. The high energy field provided to the V-Bank filter and filter media may allow for a less dense filter media to capture smaller particles based on clumping effects associated with the design of the V-Bank filter and the high energy field generated inside the V-Bank filter. Because of this and because of the pre-filter, each of the filters included in filter assembly 100 may have an increased usable lifespan. HEPA filters also offer higher resistance as compared to V-Bank filters that use lesser dense filter media. This means that a pressure drop associated with such a V-Bank filter can approach almost a quarter of the pressure drop experienced when denser HEPA filters are used. This means that a filter system built in a manner consistent with the present disclosure may filter as or more effectively than a HEPA filtration system while providing benefits of less pressure drop and/or lower energy use. For example, at a time of installation, a HEPA system may experience a pressure drop of 1.0 inches of Mercury as compared a pressure drop of 0.25 to 0.30 inches of Mercury of a DFS or EEF V-Bank filtration system.

Here the filter media fibers of filter elements are continually being exposed to the high energy field that create microbiostatis effects in the filter media. The result, depending on the efficiency of the traditional media used, is as follows: much higher particulate efficiency than traditional media filters and with fan-powered machines, up to a 99.99% at 0.002-micron filtration efficiency, with a greater gram holding weight capacity, resulting in a greater lifetime performance and less maintenance and energy cost. The technology has been proven to enable a penetration reduction of 2-3 orders of magnitude. In certain instances, HEPA or other denser filter media may be used in a V-Bank filtration system, this however, may increase energy costs because of the greater pressure drops associated with use of higher density filters.

As discussed above, contact pad 165 is located on an exterior surface of V-Bank filter 140. Contact pad 165 may be configured to directly contact a high-voltage contact included in filter assembly 105. This allows power to be coupled from power control unit 110 to the V-Bank filter via contact pad 165 without a person touching power interconnections.

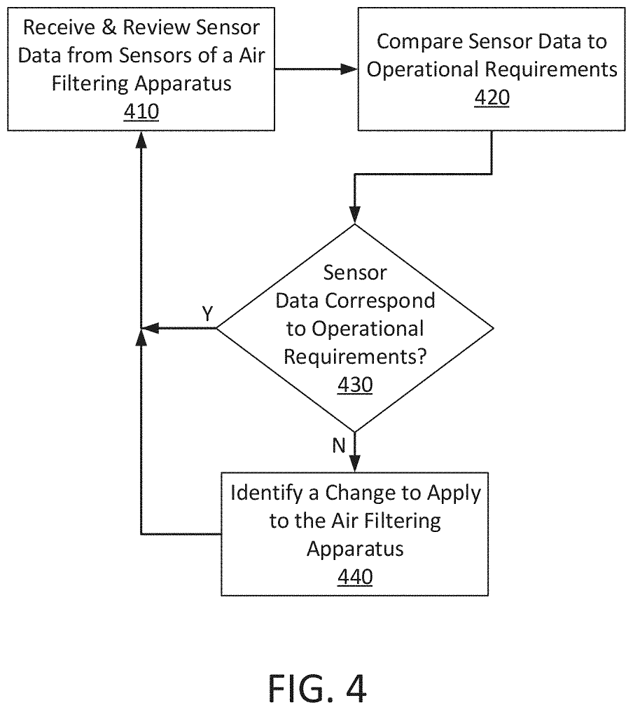

During the filtration process, 0.0027-micron and above substances may be captured and degraded with an electric field generated by an element or elements (e.g. wires 145) that is provided a voltage. In certain instances, a voltage applied to wires 145 may be varied from seven thousand volts (5 KV) to 18 KV. This high voltage may charge particles in an air flow and electric charge may be transferred to a high energy transfer grid located at one side of a filter element. High-energy transfer grids may cover 95% of an area of a filter media, only slightly increasing the resistance of the V-Bank filter 140. A V-Bank filter may include a number of rear ground control grids 170. In the V-Bank filter 140 of FIG. 4, there are eight rear ground control grids 170, one located on an exit side of each filter element on opposite sides of a V-Bank filter bank. The side view of V-Bank filter 140B includes four V-Bank filter banks. Each of these filter banks may have a first filter element and a second filter element, where a first (top) part of the first filter element and a first (top) part of the second filter element are separated by a first distance that is greater than a second distance that separates a second (bottom) part of the first filter element and a second (bottom) part of the second filter element. Because of this, each of the filter elements included in a bank of filter elements may be arranged in a V like shape. Rear control grids on particular V-Bank filter elements may be glued to the filter media and may be grounded by an electrical connection to ground bar 160. The rear ground control grid 170 helps to contain particles in the media. A filter element included in V-Bank filter 140 may include filter media sandwiched between a high energy transfer grid and a rear control grid 170. Grounding strap 135 and ground bar 160 may be electrically coupled to Earth ground via ground contact 130. The rear ground control grid 170 may also eliminate electrostatic field effects in areas outside the filter media because rear ground control grid 170 is electrically coupled to Earth ground. Read ground control grid 170 may therefore prevent electrostatic fields and any charged particles from exiting the V-Bank filter. Note that high energy wires 145 are located near an air input of the V-Bank filter and the rear control grid 170 is located near an air output of the V-Bank filter.

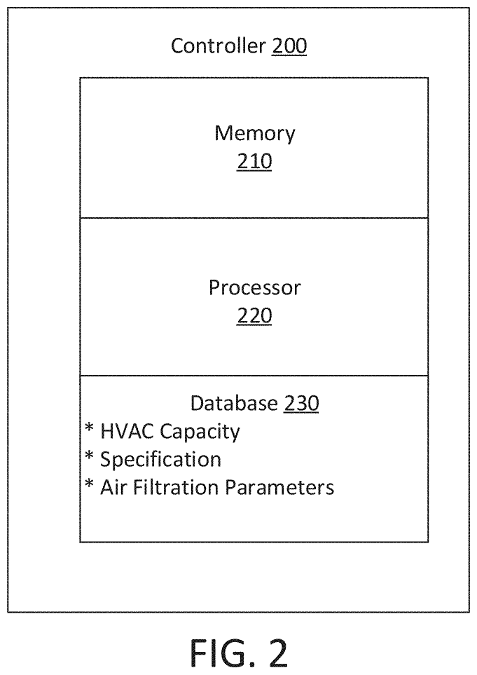

FIG. 2 includes a controller that may be used to monitor air quality, contaminates in the air (e.g. smoke, particulate matter, bacteria, fungi, or viruses), levels of volatile organic compounds (VOCS), air particle counts, air flow rates, ozone levels, and/or pressure drops when voltages applied to disinfecting filters are controlled or adjusted. Controller 200 of FIG. 2 includes memory 210, processor 220, and database(s) 230. Database(s) 230 may include one or more non-volatile data storage devices from which data may be retrieved by processor 220. Database(s) 230 may include one or more physically different database units that store data. This stored data may identify a capacity of an HVAC system, specifications of the HVAC system, parameters that affect air filtration, and/or other data.

Controller 200 may be configured using a set of settings that configure the high energy field parameters for particular applications. Configurations of these parameters may be set based on information regarding air quality standards needed for the given application. Parameters used to filter air of a commercial office building may be different from parameters used to filter air of a hospital or clean room.

Settings or sets of parameters may be stored a database that stores application parameters (i.e. an application parameter database), data that identifies the air handling capabilities of particular HVAC systems or air volume requirements of a building may be stored at a HVAC capacity database, and the operating parameter ranges of the specific DFS equipment being used in an application may be stored at a DFS specification database. Computers used to configure a controller of a DFS filter apparatus may be located in a number of different places depending upon a particular design. For example, a configuration computer could be connected to a DFS apparatus via a network such that the DFS apparatus may be controlled remotely by an end user or other entity. Alternatively, operations of a configuration computer may be implemented at a mobile device, such as a tablet or specialized industrial computing device, that allows a service/installation technician to configure a DFS system or apparatus. Such control computers may be integrated directly into a DFS control unit to give a user or technician direct access to the DFS controller 200.

A computer that sets configurations of a DFS apparatus may include an application program or program code that allows a user, an administrator, or a technician to provide settings of the DFS apparatus for a particular application. Such a software module or set of program code may identify parameters for particular application environments. Such applications may include, for example, a clean room, an office, a hospital, a research lab, a smoking room, an interior of a particular type of vehicle (e.g. an airplane, bus, car, or ship).

Parameters associated with a particular application may include a number of times per hour the air in the space needs to be cycled or an amount of air filtration requirements. The capacity of an HVAC system may be identified from a set of HVAC capacity data. This HVAC capacity data may identify a measure of cubic feet per minute (CFM) of a particular HVAC system. The CFM of a HVAC system of a building combined with data that identifies an air volume of the building may be used to identify the number of times per hour that the air included in the building can be filtered.

A processor that accesses air filtration requirement data, HVAC capacity data, or other data associated with air filtration may allow that processor to control operation of an HVAC system to meet the requirements of an application as operating conditions associated with air quality change over time. Sensors, not illustrated in FIG. 2, may sense data that is provided to processor 220 when that processor executes instructions out of a memory to control an amount of voltage provided to a charging wire or an amount of air flow. The processor may identify an amount of filtration needed to meet the requirements of a given application. This may include the processor accessing data that identifies air filtration capabilities for different levels of high energy voltage or charging capabilities.

Sensors may sense data that may be evaluated by the processor to identify various operating conditions of an air filtration system. Changes in various operating conditions of an air filtration system may allow a processor to change how the air filtration system operates as air quality changes. Sensors may monitor pressures from which pressure drops (changes in pressure from an input to an output of an air filter) may be identified. These sensors may also measure amounts of ozone generated inside of or emitted from an DFS filtration apparatus, air particle counts, and/or levels of pathogens (i.e. bacteria, fungi, or virus contaminates) in the air.

These sensors may be located at various different locations within an air filtration apparatus or remotely from the air purification system. For example, sensors may be located near an input of air filtration apparatus 100 of FIG. 1 or may be located in a room of a building remotely from the air purification system (i.e. on a wall opposite side of the room from the air purification system). Alternatively, or additionally, sensors may be located after pre-filter 115, inside of V-Bank filter 140, or at an output of filter apparatus 100 of FIG. 1. Because of this, sensor data may allow processor 220 to monitor conditions within a building, conditions inside of a filter apparatus, and conditions associated with air exiting the filter apparatus. In an instance when sensor data indicates that air in a building is ladened with smoke, voltages provided to wires 145 of FIG. 1 may be changed from 5 KV to 18 KV in order to increase a rate of particle clumping and/or air filtering. Once levels of smoke particles reduce below a threshold level, the voltage may be reduced back to 5 KV. In an instance when a particular voltage generates ozone concentrations above a threshold level, voltage applied to wires 145 may be reduced.

An application parameters database may store filtration/air quality parameters for a given application including. This data may identify a number of air changes per hour ACH, a volume a space, particle filtration data, and organism filtration parameters. These parameters can come from an air quality standard, such as EPA PM2.5 or PM1, or they can be criteria chosen by an end user.

A HVAC capacity database may store data that identifies specifications for an HVAC system being used in a given application. This may include blower motor capabilities (max CFM), fan filter coverage, or other specification data. This data may be used to identify an amount of charge (or related voltage) that needs to be introduced to a filter media to meet application parameters without exceeding capabilities of the HVAC system. This specification data may allow a processor to identify and avoid exceeding the capabilities of a blower motor or excessive pressure drops.

Data stored at database 230 may include specifications of a particular DFS system being used in a given application. This specification data may identify range of voltages (e.g. 7 KV to 18 KV) that a controller of the DFS can provide. This specification data may identify acceptable ranges of ozone levels and identify pressure drops expected at different air flow rates. Processor 220 may access this specification data to identify sets of expected operating conditions of the air filter apparatus 100 of FIG. 1. The processor may compare data from sensors and perform evaluations that may be used to identify whether an air filter apparatus is working properly. As such, controller 200 of FIG. 2 may continuously monitor and adjust operation of an air filter apparatus while verifying proper operation of that air filter apparatus. Processor 220 may send operational data or warning messages to a computer of a person responsible for managing or maintaining operation of an air filter apparatus.

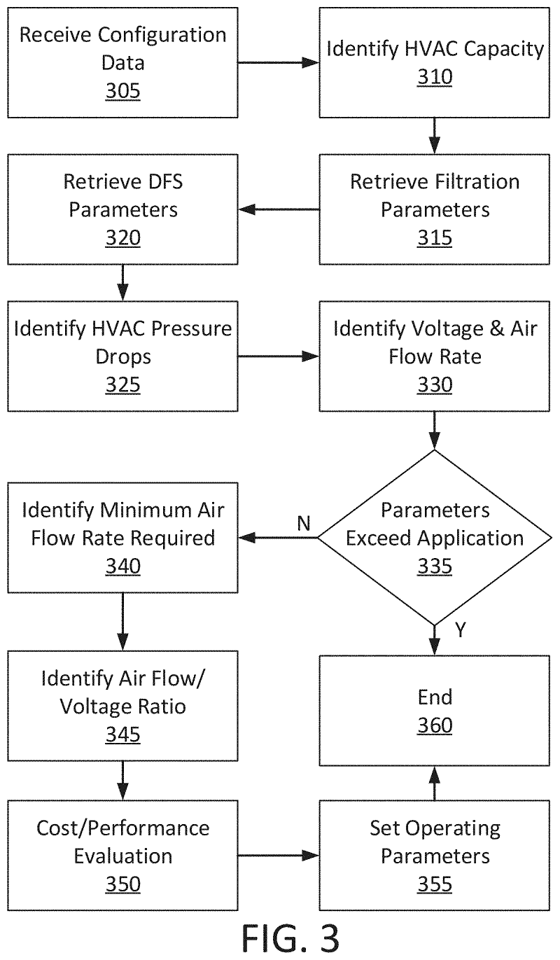

FIG. 3 illustrates a series of steps that may be performed by a processor of an air filtration apparatus when operational characteristics of the air filtration apparatus are controlled over time. The steps of FIG. 3 may include instructions of a set of program code that may be referred to as a configuration software module. Such sets of program code may allow a user, an administrator, or technician to optimize the operating parameters of a DFS or an entire HVAC system for a given application.

FIG. 3 begins with step 305 where data that configures operation of an air filtration apparatus is received or accessed. Configuration data may be received from a user that inputs that data into a user interface or may be accessed by retrieving data from a database. This configuration data may identify a volume of a building, a type of filtration apparatus, and/or an application that the type of filtration apparatus will be used in. Next in step 310, a capacity of an air filtering HVAC system being used may be identified. This may include accessing a database that stores capabilities of the type of filtration apparatus identified in the configuration data. This capacity data may identify a maximum CFM capacity of a blower, pressure drops associated with air flow rates, filtering capabilities (e.g. particle capture capabilities, a capacity to remove volatile organic compounds (VOCs), and/or pathogen capture capabilities), and voltages that may be adjusted to change the filtering capabilities of a disinfecting portion of an HVAC system.

Parameters for the given application identified in the configuration data may then be retrieved from an application parameter database in step 315 of FIG. 3. Parameters for a given application may identify a size of a particle or an organism size that the filter apparatus must remove from the air. Filtration parameters for a given application may identify a particle size of 0.002-microns and a number of air changes per hour that are best suited for a given application. After step 315, operating parameters of a disinfecting portion of the air filtration apparatus may be retrieved in step 320. These operating parameters may identify a range of voltage that can be provided to energize the disinfecting portion of the air filtration apparatus when a particular type of filter media is used. Next in step 325 pressure drops at differing air flow rates may be identified. This pressure drop data may identify a maximum pressure drop the HVAC system is capable of handling while maintaining efficient operation.

Next in step 330, an air flow rate necessary to provide a number of air changes per hour may be identified. Determination step 335 may then compare a pressure drop caused by the disinfecting portion of the air filter apparatus with an HVAC pressure drop handling capability. Determination step 335 may identify whether that pressure drop exceeds a capacity of the HVAC system. When determination step 335 identifies that the capacity of the HVAC system is or will be exceeded, program flow may move to step 360 where program flow may end. Step 360 may also include providing a message to a user indicating that the type of air filtration apparatus identified in the configuration data is not well suited for the identified application.

When determination step 335 of FIG. 3 identifies that the HVAC system is suited for the identified application, program flow may move to step 340 where a minimum air flow rate needed to achieve a required air change rate for the application may be identified in step 340. Step 340 may also identify a voltage that should be used to meet a filtering efficiency at the air flow rate identified in step 340. This voltage may correspond to a particle size filtering capability, other filtering capabilities discussed above, or particles counts that the voltage helps trap in a filter.

A range of air flow rates and disinfecting voltage settings may be evaluated to identify an initial voltage to apply to a disinfecting portion of the air filter apparatus in step 345. Next in step 350 costs and performance efficiencies associated with the air filtering (HVAC) system may be identified. This may identify where particular settings of the air filtering apparatus may result in a loss of efficiency of the HVAC system. The evaluations performed in FIG. 3 may allow the air filtering apparatus and the HVAC system to operate at a lowest air flow rate (CFM) while still meeting filtering requirements.

Costs associated with operating an air filtering system increase with energy use and these costs will tend to increase when air filters become filled with particles removed from the air over time. Because of this, a life expectancy of these air filters could also be considered as a means of identifying operating costs over time. Other factors that may affect the cost or performance of an air filtering apparatus may be associated with controlling odors and may include controlling an amount of ozone generated at a disinfecting portion of an air filtering apparatus.

When no odor control function is needed for a given application, the air filtering apparatus may be adjusted to minimize or prevent the generation of ozone instead of eliminating odors. In instances when applications odor control is required greater levels of ozone generation may be allowed. Such controls may ensure that a system does not generate or emit ozone above a given threshold level (e.g. 0.05 ppm of ozone in a volume of air). Such levels may be set based on guidelines set by the environmental protection agency (EPA). An example of an application where odor control may be important is when filtering air from a space that is a designated smoking or vaping area.

After step 350, program flow may move to step 355 where a set of operating parameters are set at a controller of the air filtering apparatus. Once the operating parameters of the air filtering apparatus have been set in step 355, program flow may end in step 360 of FIG. 3.

The steps of FIG. 3 may be used to identify whether a particular type of filtering apparatus is suitable for a given application for a building of a given volume. Evaluations may be performed where a first set of parameters may be associated with air of a clean room is being filtered and a second set of parameters may be evaluated when air of an office is being filtered. The steps of FIG. 3 may be used to identify settings that may be used to achieve best results for a given set of customer requirements. A standard air filtration apparatus may be sold to either to a customer building a clean room or to a customer installing an air filter apparatus in an office building. In such an instance, all a customer need do is to provide a set of configuration data and allow the processor to perform evaluations that result in selection of an appropriate type of air filter apparatus configuration.

FIG. 4 illustrates a series of steps that may be performed when operation of a disinfection filtration system (DFS) or filter apparatus is controlled. The Steps of FIG. 4 may be performed by when the processor 220 executes instructions out of memory 210 of the controller 200 of FIG. 2. FIG. 4 begins with a first step 410 where sensor data is received and reviewed. As mentioned above, these sensors may monitor pressure, amounts of generated ozone, VOC levels, air particle counts, and/or levels of pathogens in the air. Next in step 420 of FIG. 4, the sensor data may be compared to operational requirements of an air filtration apparatus. Data from different pressure sensors may be used to identify pressure drops in an air filter apparatus, to identify levels of ozone generated in or emitted by an air filter apparatus, or to identify whether the air filter apparatus is removing particles and/or pathogens from the air in a manner that meets a set of operational requirements.

Determination step 430 may then identify whether operation of the air filtering apparatus corresponds to the operational requirements, when yes program flow may move back to step 410 where additional sensor data is received and evaluated. When determination step 430 identifies that the sensor data does not correspond to operational requirement, program flow moves to step 440 where a change is applied to the air filtering apparatus. As mentioned above, changes applied to an air filtering apparatus may include changing an air flow rate or changing a voltage of a disinfecting portion of the air filtering apparatus based on a set of requirements.

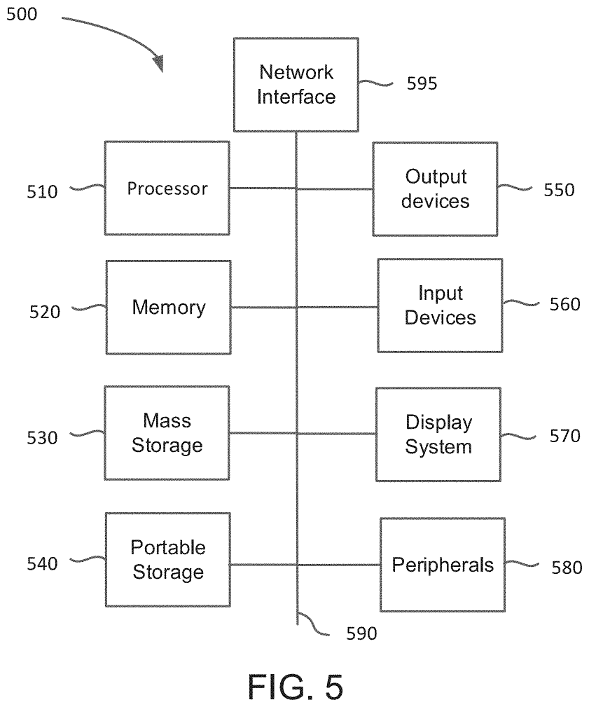

FIG. 5 illustrates a computing system that may be used to implement an embodiment of the present invention. The computing system 500 of FIG. 5 includes one or more processors 510 and main memory 520. Main memory 520 stores, in part, instructions and data for execution by processor 510. Main memory 520 can store the executable code when in operation. The system 500 of FIG. 5 further includes a mass storage device 530, portable storage medium drive(s) 540, output devices 550, user input devices 560, a graphics display 570, peripheral devices 580, and network interface 595.

The components shown in FIG. 5 are depicted as being connected via a single bus 590. However, the components may be connected through one or more data transport means. For example, processor unit 510 and main memory 520 may be connected via a local microprocessor bus, and the mass storage device 530, peripheral device(s) 580, portable storage device 540, and display system 570 may be connected via one or more input/output (I/O) buses.

Mass storage device 530, which may be implemented with a magnetic disk drive or an optical disk drive, is a non-volatile storage device for storing data and instructions for use by processor unit 510. Mass storage device 530 can store the system software for implementing embodiments of the present invention for purposes of loading that software into main memory 520.

Portable storage device 540 operates in conjunction with a portable non-volatile storage medium, such as a FLASH memory, compact disk or Digital video disc, to input and output data and code to and from the computer system 500 of FIG. 5. The system software for implementing embodiments of the present invention may be stored on such a portable medium and input to the computer system 500 via the portable storage device 540.

Input devices 560 provide a portion of a user interface. Input devices 560 may include an alpha-numeric keypad, such as a keyboard, for inputting alpha-numeric and other information, or a pointing device, such as a mouse, a trackball, stylus, or cursor direction keys. Additionally, the system 500 as shown in FIG. 5 includes output devices 550. Examples of suitable output devices include speakers, printers, network interfaces, and monitors.

Display system 570 may include a liquid crystal display (LCD), a plasma display, an organic light-emitting diode (OLED) display, an electronic ink display, a projector-based display, a holographic display, or another suitable display device. Display system 570 receives textual and graphical information and processes the information for output to the display device. The display system 570 may include multiple-touch touchscreen input capabilities, such as capacitive touch detection, resistive touch detection, surface acoustic wave touch detection, or infrared touch detection. Such touchscreen input capabilities may or may not allow for variable pressure or force detection.

Peripherals 580 may include any type of computer support device to add additional functionality to the computer system. For example, peripheral device(s) 580 may include a modem or a router.

Network interface 595 may include any form of computer interface of a computer, whether that be a wired network or a wireless interface. As such, network interface 595 may be an Ethernet network interface, a BlueTooth™ wireless interface, and 802.11 interface, or a cellular phone interface.

The components contained in the computer system 500 of FIG. 5 are those typically found in computer systems that may be suitable for use with embodiments of the present invention and are intended to represent a broad category of such computer components that are well known in the art. Thus, the computer system 500 of FIG. 5 can be a personal computer, a hand held computing device, a telephone (“smart” or otherwise), a mobile computing device, a workstation, a server (on a server rack or otherwise), a minicomputer, a mainframe computer, a tablet computing device, a wearable device (such as a watch, a ring, a pair of glasses, or another type of jewelry/clothing/accessory), a video game console (portable or otherwise), an e-book reader, a media player device (portable or otherwise), a vehicle-based computer, some combination thereof, or any other computing device. The computer can also include different bus configurations, networked platforms, multi-processor platforms, etc. The computer system 500 may in some cases be a virtual computer system executed by another computer system. Various operating systems can be used including Unix, Linux, Windows, Macintosh OS, Palm OS, Android, iOS, and other suitable operating systems.

The present invention may be implemented in an application that may be operable using a variety of devices. Non-transitory computer-readable storage media refer to any medium or media that participate in providing instructions to a central processing unit (CPU) for execution. Such media can take many forms, including, but not limited to, non-volatile and volatile media such as optical or magnetic disks and dynamic memory, respectively. Common forms of non-transitory computer-readable media include, for example, a FLASH memory/disk, a hard disk, magnetic tape, any other magnetic medium, a CD-ROM disk, digital video disk (DVD), any other optical medium, RAM, PROM, EPROM, a FLASH EPROM, and any other memory chip or cartridge.

The functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

While various flow diagrams provided and described above may show a particular order of operations performed by certain embodiments of the invention, it should be understood that such order is exemplary (e.g., alternative embodiments can perform the operations in a different order, combine certain operations, overlap certain operations, etc.).

The foregoing detailed description of the technology herein has been presented for purposes of illustration and description. It is not intended to be exhaustive or to limit the technology to the precise form disclosed. Many modifications and variations are possible in light of the above teaching. The described embodiments were chosen in order to best explain the principles of the technology and its practical application to thereby enable others skilled in the art to best utilize the technology in various embodiments and with various modifications as are suited to the particular use contemplated. It is intended that the scope of the technology be defined by the claim.

What is claimed is:

1. A method for controlling an air filtration apparatus, the method comprising:
receiving sensor data;
evaluating the received sensor data to identify a first set of operating conditions associated with the air filtration apparatus;
accessing data that identifies operational requirements of an operating environment associated with the air filtration apparatus;
identifying that an operational setting of the air filter apparatus should be changed based on identifying that the first set of conditions do not correspond to the operational requirements of the operating environment; and
initiating a change to the operational setting of the air filtration apparatus, wherein the change to the operational setting results in changing the first set of operating conditions associated with the air filtration apparatus.

2. The method of claim 1, further comprising:
identifying the environment; and
accessing data that identifies the requirements of the environment.

3. The method of claim 1, further comprising:
identifying a particle count included in air at an input of the air filtration apparatus; and
identifying a voltage to apply to a high energy element of the air filtration apparatus, wherein the operational setting of the air filter apparatus is a voltage setting and the change to the operational setting results in the voltage applied to the high energy element being changed to the identified voltage.

4. The method of claim 1, further comprising:
identifying a contaminate level included in air at an input of the air filtration apparatus; and
identifying a voltage to apply to a high energy element of the air filtration apparatus, wherein the operational setting of the air filter apparatus is a voltage setting and the change to the operational setting results in the voltage applied to the high energy element being changed to the identified voltage.

5. The method of claim 1, further comprising:
identifying a level of ozone at a part of the air filtration apparatus; and
identifying a voltage to apply to a high energy element of the air filtration apparatus, wherein the operational setting of the air filter apparatus is a voltage setting and the change to the operational setting results in the voltage applied to the high energy element being changed to the identified voltage.

6. The method of claim 1, air flow change

7. The method of claim 1, wherein the received sensor data includes data that identifies a level of pathogens included in air.

8. The method of claim 7, wherein the pathogens include at least one of a bacteria, a fungus, or a virus.

9. A non-transitory computer-readable storage medium having embodied thereon a program executable by a processor for implementing a method for controlling an air filtration apparatus, the method comprising:
receiving sensor data;
evaluating the received sensor data to identify a first set of operating conditions associated with the air filtration apparatus;
accessing data that identifies operational requirements of an operating environment associated with the air filtration apparatus;
identifying that an operational setting of the air filter apparatus should be changed based on identifying that the first set of conditions do not correspond to the operational requirements of the operating environment; and
initiating a change to the operational setting of the air filtration apparatus, wherein the change to the operational setting results in changing the first set of operating conditions associated with the air filtration apparatus.

10. The non-transitory computer-readable storage medium of claim 9, further comprising:
identifying the environment; and
accessing data that identifies the requirements of the environment.

11. The non-transitory computer-readable storage medium of claim 9, further comprising:
identifying a particle count included in air at an input of the air filtration apparatus; and
identifying a voltage to apply to a high energy element of the air filtration apparatus, wherein the operational setting of the air filter apparatus is a voltage setting and the change to the operational setting results in the voltage applied to the high energy element being changed to the identified voltage.

12. The non-transitory computer-readable storage medium of claim 9, further comprising:
identifying a contaminate level included in air at an input of the air filtration apparatus; and
identifying a voltage to apply to a high energy element of the air filtration apparatus, wherein the operational setting of the air filter apparatus is a voltage setting and the change to the operational setting results in the voltage applied to the high energy element being changed to the identified voltage.

13. The non-transitory computer-readable storage medium of claim 9, further comprising:
identifying a level of ozone at a part of the air filtration apparatus; and
identifying a voltage to apply to a high energy element of the air filtration apparatus, wherein the operational setting of the air filter apparatus is a voltage setting and the change to the operational setting results in the voltage applied to the high energy element being changed to the identified voltage.

14. The non-transitory computer-readable storage medium of claim 9, air flow change

15. The non-transitory computer-readable storage medium of claim 9, wherein the received sensor data includes data that identifies a level of pathogens included in air.

16. The non-transitory computer-readable storage medium of claim 15, wherein the pathogens include at least one of a bacteria, a fungus, or a virus.

17. An apparatus for controlling an air filtration apparatus, the apparatus comprising:
a plurality of sensors that sense data;
a memory; and
a processor that executes instructions out of the memory to:
evaluate the received sensor data to identify a first set of operating conditions associated with the air filtration apparatus,
access data that identifies operational requirements of an operating environment associated with the air filtration apparatus,
identify that an operational setting of the air filter apparatus should be changed based on identifying that the first set of conditions do not correspond to the operational requirements of the operating environment, and
initiate a change to the operational setting of the air filtration apparatus, wherein the change to the operational setting results in changing the first set of operating conditions associated with the air filtration apparatus.

18. The apparatus of claim 1, wherein the processor also executes the instructions out of the memory to:
identify the environment, and
access data that identifies the requirements of the environment.

19. The apparatus of claim 1, wherein the processor also executes the instructions out of the memory to:
identify a particle count included in air at an input of the air filtration apparatus; and
identify a voltage to apply to a high energy element of the air filtration apparatus, wherein the operational setting of the air filter apparatus is a voltage setting and the change to the operational setting results in the voltage applied to the high energy element being changed to the identified voltage.

20. The apparatus of claim 1, wherein the processor also executes the instructions out of the memory to:
identify a contaminate level included in air at an input of the air filtration apparatus; and
identify a voltage to apply to a high energy element of the air filtration apparatus, wherein the operational setting of the air filter apparatus is a voltage setting and the change to the operational setting results in the voltage applied to the high energy element being changed to the identified voltage.
